(19) United States
(12) Patent Application Publication
(10) Pub. No.: US 2015/0105329 A1
(43) Pub. Date: Apr. 16, 2015

(54) Methods and Pharmaceutical Compositions for Prevention or Treatment of Ischemia Related Organ Damage

(71) Applicants: INSERM (Institut National de la Sante et de la Recherche Medicale), Paris (FR); Universite de Sherbrooke, Sherbrooke (CA); Universite Paris Descartes, Paris (FR); Universite Pierre et Marie Curie - Paris IV, Paris (FR); Assistance Publique - Hopitaux de Paris, Paris (FR); Universite Paris VII (Denis Diderot), Paris (FR)

(72) Inventors: Francois Alhenc-Gelas, Paris (FR); Nadine Bouby, Paris (FR); Fernand Junior Gobeil, Sherbrooke (CA); Ronan Roussel, Paris (FR); Ludovic Waeckel, Paris (FR); Louis Potier, Paris (FR)

(21) Appl. No.: 14/387,680

(22) PCT Filed: Mar. 26, 2013
(86) PCT No.: PCT/EP2013/056347

§ 371 Date: Sep. 24, 2014

(30) Foreign Application Priority Data
Mar. 26, 2012 (EP) 12305349.8

(51) Int. Cl. A61K 38/04

(52) U.S. Cl. CPC A61K 38/043
USPC 514/20.6; 435/7.21

(57) ABSTRACT

The present invention relates to methods and compositions for the prevention or treatment of ischemia related organ damage.

### FIELD OF THE INVENTION

The present invention relates to use of the agonists of the bradykinin B2 receptor for the prevention or treatment of ischemia related organ damage.

### BACKGROUND OF THE INVENTION

Kinins, are peptides that are released in blood and tissues from kininogen precursors by the action of a group of serine proteases, called kallikreins. Active kinins are very labile oligopeptides, with half-lives in the range of a few seconds to a few minutes, due to enzyme degradation and/or rapid kidney clearance (Leeb-Lundberg, L. M.; L. Pharmacol Rev, 2005, 57, 27-77). Several tissue and circulating peptidases can inactivate kinins. The main kinin inactivating enzyme in the circulation is the dipeptidylcarboxypeptidase A also known as angiotensin I-converting enzyme (ACE) or kininase II.

Kinins are involved in a wide range of biological processes through two different G protein-coupled seven transmembrane domains receptors, called B1 and B2 (Regoli, D.; Biol Chem, 2001, 382, 31-5. Leeb-Lundberg, L. M.; L. Pharmacol Rev, 2005, 57, 27-77). The B2 receptor is the main kinin receptor and mediates most of the known physiological action of kinins. This receptor, contrary to the B1 receptor, is constitutively synthesized in target organs, more particularly in the endothelia, smooth muscles, epithelia and neurons.

From the endothelia, kinins promote the release of nitric oxide (NO) and of prostacyclin (PGI₂), two potent vasodilators and anti-thrombotic agents that mediate the arterial dilatation, which is required to maintain optimal blood flow to tissues and thus sustain their basic vital functions. Kinins also release plasminogen activators and trigger fibrinolysis. Another vascular-related target for kinins is a subset of bone narrow and circulating endothelial progenitor cells with neovessel development promoting capacity.

Ischemia is a reduction in blood flow, a restricted or insufficient supply in blood to an organ, generally due to a constriction or obstruction of a blood vessel. This reduction may occur for a variety of reasons, including but not limited to thrombosis, embolism, aneurysm, spasm, rupture or collapse of a blood vessel. Ischemia results in tissue damage or dysfunction because of a lack of oxygen and nutrients. Ischemia affects almost all organs and tissues such as but not limited to cardiac ischemia, cerebral ischemia, retinal ischemia, limb ischemia, kidney ischemia.

However, there is a need to develop new drugs that will be suitable for treatment of ischemia related organ damage. In this way, it has been suggested that characterisation of new therapeutic targets in ischemia related organ damage may be highly desirable.

Experimental studies have shown that the kallikrein-kinin system plays an important role in ischemia-induced organ damage. Indeed, a beneficial effect of endogenous kinins in the recovery of the ischemic heart has been evidenced on the basis of pharmacological experiments using ACE inhibitors, with or without pretreatment with the kinin B2-receptor antagonist HOE140 (Hartman J C, J Cardiovasc Pharmacol, 1993, 21: 996-1003; Goto M, Circ Res, 1995, 77: 611-21; Griol-Charbhili, FASEB J, 2005, 19(9):1172-4). Loss of the infarct size reducing effect of ACE inhibitors is also observed in mice deficient in the B2 receptor (Yang X P, Hypertension 1997; Griol-Charbhili, FASEB J, 2005, 19(9):1172-4). Moreover, the kallikrein-kinin system is involved in reactive angiogenesis in experimental peripheral ischemic disease in the mouse (Stone, O. Arterioscler Thromb Vasc Biol, 2009, 29, 657-64). ACE inhibitors have been shown to enhance reactive angiogenesis in this model and this effect appears to be for a large part kinin and B2 receptor-dependent (Ebrahimian, T. G.; Arterioscler Thromb Vasc Biol, 2005, 25, 65-70. Silvestre, J. S.; Circ Res, 2001, 89, 678-83.)

But until now, there is no disclosure in the art of the effect of neither B2-receptor agonists on infarct size in cardiac ischemia, nor the pro-angiogenic effect of B2-receptor agonists, nor the use of B2-receptor agonist in the treatment of ischemia related organ damage.

### SUMMARY OF THE INVENTION

The present invention relates to a compound which is selected from the group consisting of B2-receptor agonist, B2-receptor expression activator, kinin expression activator, kininogen expression activator, kallikreins expression activator, kininase expression inhibitor or kininase inhibitor for use in the prevention or treatment of ischemia related organ damage in a subject in need thereof.

### DETAILED DESCRIPTION OF THE INVENTION

The role of B2-receptor agonists in the ischemia was investigated by the inventors by measuring infarct size in cardiac ischemia and exploring pro-angiogenic effect of B2-receptor agonist in diabetic mice submitted to femoral occlusion. The inventors found that B2-receptor agonists reduce infarct size in cardiac ischemia reperfusion injury. The inventors also demonstrated that B2-receptor agonist induced pro-angiogenic effect and restored downstream blood flow in diabetic mice submitted to femoral occlusion.

### Therapeutic Methods and Uses

The present invention relates to a compound which is selected from the group consisting of B2-receptor agonist, B2-receptor expression activator, kinin expression activator, kininogen expression activator, kallikreins expression activator, kininase expression inhibitor or kininase inhibitor for use in the prevention or treatment of ischemia related organ damage in a subject in need thereof.

As used herein, the term “subject” denotes a mammal. In a preferred embodiment of the invention, a subject according to the invention refers to any subject (preferably human) susceptible of having or afflicted with ischemia related organ damage.

The method of the invention may be performed for ischemia related organ damage in any type of ischemia such as revised in the World Health Organisation Classification of ischemia and selected from the group: Cardiac ischemia, Ischaemic heart diseases (120-125 groups): Angina pectoris, Acute myocardial infarction, Subsequent myocardial infarction, Certain current complications following acute myocardial infarction (such as Haemopericardium, Atrial septal defect, Ventricular septal defect, Rupture of cardiac wall without haemopericardium, Rupture of chordae tendineae, Rupture of papillary muscle, Thrombosis of atrium, auricular appendage, and ventricle), Other acute ischaemic heart diseases (such as Coronary thrombosis not resulting in myocardial infarction and Dressler's syndrome), Chronic ischaemic heart disease (such as Atherosclerotic cardiovascular disease, Atherosclerotic heart disease, Old myocardial infarction, Ischaemic cardiomyopathy, Silent myocardial ischaemia); Brain ischemia, Transient cerebral ischaemic attacks and related syndromes (G45 group): Vertebro-basilar artery syndrome, Carotid artery syndrome (hemispheric), Multiple and bilateral precerebral artery syndromes, Amaurosis fugax, Transient global amnesia; Retinal ischemia; Kidney ischemia; intestinal ischemia; Limb ischemia; peripheral or diabetic-related ischemia; lung ischemia; liver ischemia; mesenteric ischemia; ischemia-reperfusion or ischemia-reperfusion organ damage.

The term “kinin” has its general meaning in the art and refers to bradykinin and lysil-bradykinin. Kinin, bradykinin and lysil-bradykinin refer to endogenous nona- and deca-peptide that are generated by cleavage of the precursor polypeptide (kininogen) by specific proteases (kallikreins) within numerous tissues of the body (Regoli, D. and Barabe, J. Pharmacol. Rev., 1980, 32, 1-46; Hall, J. M., Pharmacol. Ther., 1992, 56, 131-190; Leeb-Lundberg et al., Pharmacol. Rev. 2005, 57: 27-77). Certain enzymes of the kininase family degrade bradykinin and related peptides and thus inactivate these peptides. Kinins exert their actions through two different G protein-coupled seven transmembrane domains receptors, called B1 and B2.

The term “kininogen” has its general meaning in the art and refers to polypeptide, precursor for the kinin.

The term “Kallikreins” has its general meaning in the art and refers to specific protease responsible of the generation of kinin by the cleavage of the precursor polypeptide (kininogen).

The term “kininase” has its general meaning in the art and refers to enzymes responsible of kinin and related peptides degradation and thus their inactivation.

The term “B2-receptor” has its general meaning in the art and refers to kinin receptor type B2 or bradykinin receptor type B2 such as the B2-receptor expressed in endothelial cell.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs which are modified, by processes such as capping, polyadenylation, methylation, and editing, and proteins (e.g., phosphatidylserine receptor) modified by, for example, methylation, acetylation, phosphorylation, ubiquitination, SUMOylation, ADP-ribosylation, myristilation, and glycosylation.

An “activator of expression” refers to a natural or synthetic compound that has a biological effect to activate the expression of a gene.

An “inhibitor of expression” refers to a natural or synthetic compound that has a biological effect to inhibit the expression of a gene.

The term “B2-receptor agonist” or “bradykinin B2 receptor agonist” has its general meaning in the art and refers to a compound that selectively activates the B2 receptor. The term “B2-receptor agonist” refers to any compound that can directly or indirectly stimulate the signal transduction cascade related to the B2-receptor. As used herein, the term “selectively activates” refers to a compound that preferentially binds to and activates B2-receptor with a greater affinity and potency, respectively, than its interaction with the other sub-types or isoforms of the bradykinin receptor family (B1-receptor). Compounds that prefer B2-receptor, but that may also activate other bradykinin receptor sub-types, as partial or full agonists, and thus that may have multiple bradykinin receptor activities, are contemplated. Typically, a B2-receptor agonist is a small organic molecule or a peptide.

Tests and assays for determining whether a compound is a B2-receptor agonist are well known by the skilled person in the art such as described in Savard et al., 2013 Biol Chem. 2013 March; 394(3):353-60; U.S. Pat. No. 6,316,413; U.S. patent Ser. No. 12/861,941.

In one embodiment of the invention, the compound which is a B2-receptor agonist may be a peptide, such as kinin or lysyl-bradykinin or bradykinin analogue or truncated bradykinin peptide such as compounds described, for example, in U.S. patent Ser. No. 12/861,941 and the agonist named peptide 20 [Hyp(3), Thi(5), (N)Chg(7), Thi(8)]-BK (Belanger S, Peptides 2009).

In one embodiment, a B2-receptor agonist is a non-peptide compound, such as a compound described, for example, in U.S. Pat. No. 6,015,818; U.S. Pat. No. 6,127,389; U.S. Pat. No. 6,958,349; U.S. Pat. No. 6,509,366; U.S. Pat. No. 6,420,365; and U.S. Pat. No. 6,358,949.

A B2-receptor agonist also includes peptide mimetics, metabolically and/or conformationally stabilized peptide analogs, derivatives, and pseudo-peptides with one or more non-peptide bonds, especially containing D-amino acids and/or at least one non-peptide bond. Bradykinin and related peptides, and other peptides, mimetics and/or metabolically and/or conformationally stabilized peptide analogs and/or derivatives or pseudopeptides with one or more non-peptide bonds, especially containing D-amino acids and/or at least one non-peptide bond, of the invention are useful in the prevention or treatment of ischemia related organ damage.

Said B2-receptor agonist may be a pseudopeptide such as compounds described, for example, in U.S. Pat. No. 6,316,413.

In one embodiment, the compound which is a B2-receptor agonist may be a bradykinin derivatives or modified bradykinin such as compounds described, for example, in WO 89/09231, U.S. Pat. No. 5,112,596 and U.S. Pat. No. 5,268,164. Said bradykinin derivatives are obtained by reduction of one of the amide linkages such as RMP7 compound.

In a further aspect, the present invention relates to a method of screening a candidate compound for use as a drug for the prevention or treatment of ischemia related organ damage in a subject in need thereof, wherein the method comprises the steps of:
providing a B2-receptor, providing a cell, tissue sample or organism expressing a B2-receptor,
providing a candidate compound such as small organic molecule, peptide, polypeptide, non-peptide compound, peptide mimetics, metabolically and/or conformationally stabilized peptide analogs, derivatives or pseudo-peptides,
measuring the B2-receptor activity,
and selecting positively candidate compounds that induce B2-receptor activity.

The term “B2-receptor activity” has its general meaning in the art and refers to the biological activity associated with the activation of the B2-receptor resulting from its signal transduction cascade, and including any of the downstream biological effects resulting from the binding of the candidate compound to B2-receptor that may be equal or higher than the biological effect resulting from the binding of the B2-receptor to its natural ligands.

Preferably, measuring the B2-receptor activity involves determining a Ki on the B2-receptor cloned and transfected in a stable manner into a CHO cell line or measuring one or more of the second messengers of the B2-receptor (inositol phosphates (IPs), intracellular Ca²⁺ concentration [Ca²⁺]ᵢ, cGMP, cAMP) in the present or absence of the candidate compound.

Tests and assays for screening and determining whether a candidate compound is a B2-receptor agonist are well known in the art (Savard et al., 2013 Biol Chem. 2013 March; 394(3):353-60; U.S. Pat. No. 6,316,413; U.S. patent Ser. No. 12/861,941). In vitro and in vivo assays may be used to assess the potency and selectivity of the candidate compounds to induce B2-receptor activity.

Activities of the candidate compounds, their ability to bind B2-receptor and their ability to induce similar effects to those of bradykinin may be tested using isolated endothelial cells expressing B2-receptor, CHO cell line cloned and transfected in a stable manner by the human B2-receptor or blood vessels.

Activities of the candidate compounds and their ability to bind to the B2-receptor may be assessed by the determination of a Ki on the B2-receptor cloned and transfected in a stable manner into a CHO cell line and measuring one or more of the second messengers of the B2-receptor (inositol phosphates (IPs), intracellular Ca²⁺ concentration [Ca²⁺]ᵢ, cGMP, cAMP) in the present or absence of the candidate compound. The ability of the candidate compounds to induce functional effects comparable to those of bradykinin may be assessed by the determination of the pD₂, the concentration causing the B2-receptor-dependent contraction of the human umbilical vein.

Cells and blood vessels expressing another receptor than B2-receptor may be used to assess selectivity of the candidate compounds.

### Pharmaceutical Composition

The compound of the invention may be used or prepared in a pharmaceutical composition.

In one embodiment, the invention relates to a pharmaceutical composition comprising the compound of the invention and a pharmaceutical acceptable carrier for use in the prevention or treatment of ischemia related organ damage in a subject of need thereof.

Typically, the compound of the invention may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The compound of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

In one embodiment, the invention relates to a pharmaceutical composition comprising the compound of the invention and a B1-receptor agonists for use in the prevention or treatment of ischemia related organ damage in a subject of need thereof.

Pharmaceutical compositions of the invention may include any further compound which is used in the prevention or treatment of ischemia. For example, the anti-ischemia may include but are not limited to angiotensin-converting enzyme (ACE) inhibitors, angiotensin II receptor blockers, beta blockers, calcium channel blockers, acetylsalicylate, antiplatelets agents, anticlotting agents, fibrinolytic agents.

Pharmaceutical compositions of the invention may include any further compound which is used as pro-angiogenic compound.

In one embodiment, said additional active compounds may be contained in the same composition or administrated separately.

In another embodiment, the pharmaceutical composition of the invention relates to combined preparation for simultaneous, separate or sequential use in the prevention or treatment of ischemia related organ damage.

### Biomaterials

The present invention also relates to the use of the compound of the invention for the preparation of biomaterials or medical delivery devices selected for example among endovascular prostheses, such as stents, bypass grafts, internal patches around the vascular tube, external patches around the vascular tube, vascular cuff, and angioplasty catheter.

In this respect, the invention relates more particularly to biomaterials or medical delivery devices as mentioned above, coated with such compound of the invention as defined above, said biomaterials or medical devices being selected among endovascular prostheses, such as stents, bypass grafts, internal patches around the vascular tube, external patches around the vascular tube, vascular cuff, and angioplasty catheter. Such a local biomaterial or medical delivery device can be used to reduce stenosis as an adjunct to revascularizing, bypass or grafting procedures performed in any vascular location including coronary arteries, carotid arteries, renal arteries, peripheral arteries, cerebral arteries or any other arterial or venous location, to reduce anastomic stenosis such as in the case of arterial-venous dialysis access with or without polytetrafluoro-ethylene grafting and with or without stenting, or in conjunction with any other heart or transplantation procedures, or congenital vascular interventions.

For illustration purpose, such endovascular prostheses and methods for coating the compound of the invention thereto are more particularly described in WO2005094916, or are those currently used in the art. The compounds used for the coating of the prostheses should preferentially permit a controlled release of said agonist. Said compounds could be polymers (such as sutures, polycarbonate, Hydron, and Elvax), biopolymers/biomatrices (such as alginate, fucans, collagen-based matrices, heparan sulfate) or synthetic compounds such as synthetic heparan sulfate-like molecules or combinations thereof. Other examples of polymeric materials may include biocompatible degradable materials, e. g. lactone-based polyesters orcopolyesters, e. g. polylactide; polylactide-glycolide; polycaprolactone-glycolide; polyorthoesters; polyanhydrides; polyaminoacids; polysaccharides; polyphospha-zenes; poly (ether-ester) copolymers, e. g. PEO-PLLA, or mixtures thereof; and biocompatible non-degrading materials, e. g. polydimethylsiloxane; poly (ethylene-vinylacetate); acrylate based polymers or coplymers, e. g. polybutylmethacrylate, poly (hydroxyethyl methyl-methacrylate); polyvinyl pyrrolidinone; fluorinated polymers such as polytetrafluoethylene; cellulose esters. When a polymeric matrix is used, it may comprise 2 layers, e. g. a base layer in which said agonist is incorporated, such as ethylene-co-vinylacetate and polybutylmethacrylate, and a top coat, such as polybutylmethacrylate, which acts as a diffusion-control of said agonist. Alternatively, said agonist may be comprised in the base layer and the adjunct may be incorporated in the outlayer, or vice versa.

Such biomaterial or medical delivery device may be biodegradable or may be made of metal or alloy, e. g. Ni and Ti, or another stable substance when intended for permanent use. The compound of the invention may also be entrapped into the metal of the stent or graft body which has been modified to contain micropores or channels. Also internal patches around the vascular tube, external patches around the vascular tube, or vascular cuff made of polymer or other biocompatible materials as disclosed above that contain the agonist of the invention may also be used for local delivery.

Said biomaterial or medical delivery device allow the compound of the invention releasing from said biomaterial or medical delivery device over time and entering the surrounding tissue. Said releasing may occur during 1 month to 1 year. The local delivery according to the present invention allows for high concentration of the compound of the invention at the disease site with low concentration of circulating compound. The amount of said compound used for such local delivery applications will vary depending on the compounds used, the condition to be treated and the desired effect. For purposes of the invention, a therapeutically effective amount will be administered.

The local administration of said biomaterial or medical delivery device preferably takes place at or near the vascular lesions sites. The administration may be by one or more of the following routes: via catheter or other intravascular delivery system, intranasally, intrabronchially, interperitoneally or eosophagal. Stents are commonly used as a tubular structure left inside the lumen of a duct to relieve an obstruction. They may be inserted into the duct lumen in a non-expanded form and are then expanded autonomously (self-expanding stents) or with the aid of a second device in situ, e. g. a catheter-mounted angioplasty balloon which is inflated within the stenosed vessel or body passageway in order to shear and disrupt the obstructions associated with the wall components of the vessel and to obtain an enlarged lumen.

The biomaterial of the invention may be coated with any other compounds as above described for pharmaceutical compositions.

The invention will be further illustrated by the following figures and examples. However, these examples and figures should not be interpreted in any way as limiting the scope of the present invention.

### FIGURES

FIG. 1: Reducing effect of acute administration of B2 agonist on the infarct size relative to area at risk. The effect is abolished in the presence of the B2 antagonist, Hoe140. Results are expressed as means±SEM, n=4-7/group. * p<0.05 vs saline, Fisher test post ANOVA.

FIG. 2: Chronic administration of B2 agonist (30 nmol/kg·h) has a proangiogenic effect in diabetic mice. Results are expressed as means±SEM, n=6/group, t test * p<0.05, ** p<0.01 vs control.

FIG. 3: Chronic administration of B2 agonist (30 nmol/kg·h) increase capillary density analysis of diabetic mice.

### EXAMPLES

The experiment described above were done with the agonist named peptide 20 [Hyp(3), Thi(5), (N)Chg(7), Thi(8)]-BK in ref (Belanger S, Peptides 2009). This compound exhibits greater in vitro affinities and potencies than bradykinin at the naturally expressed and recombinant hB2R. Its potency and duration of action in vivo is highly superior to bradykinin thus inferring that it can withstand intravascular proteolysis.

All the experiments were performed in accordance with the European Community guidelines for the care and use of laboratory animals.

### Example 1

### Effect of B2 Agonism on Myocardial Ischemia/Reperfusion Injury

Material and Methods

Ischemia/Reperfusion (IR) Protocol:

Adult C57/Bl6J mice of 12-15 weeks old (20-30 g) were anesthetized with sodium pentobarbital (60 mg/kg, i.p.). The animals were intubated and ventilated with 100% oxygen (200 μl/breath at a rate of 170 breaths/min), using a Harvard rodent ventilator (Model 845, Harvard Apparatus, Les Ulis, France). Body temperature was monitored with a rectal probe connected to a digital thermometer, and maintained at 37° C. using a heating pad. A catheter was inserted into the jugular vein for bolus injection of drug (0.01 ml/10 g BW, 10-20 sec). The electrocardiogram (ECG) was recorded throughout the experiments on a Gould TA240 recorder (ECG biotech; Gould Instruments, Cleveland, Ohio, USA). A left thoracotomy was performed to expose the heart, and the pericardium was removed. The left anterior descending coronary artery was occluded with an 8.0 prolene suture, 2 mm from the tip of the left atrium for 30 min. Successful coronary occlusion was verified by the development of a pale color in the distal myocardium and by ST segment elevation and QRS widening on the ECG. After 30 min of sustained ischemia, coronary blood flow was restored by loosening the suture. Successful reperfusion was confirmed by visualization of hyperaemic response and restoration of normal ECG. The lungs were then reinflated by increasing positive end expiratory pressure, and the chest was closed. Reperfusion was maintained for a 3-h period. Drugs dissolved in isotonic saline were injected 5 minutes before reperfusion. B2 receptor agonist was administrated at different non hypotensive dosages as defined in preliminary experiments. To ensure the role of specific activation of B2 receptor, B2 agonist was tested with pretreatment by B2R antagonist (HOE140).

Measurement of Infarct Size (IS):

After reperfusion, the chest was reopened, the coronary artery was reoccluded, and 0.5 ml of a 5% Evans blue solution was injected as a bolus into the jugular vein in order to delineate the area at risk (AR), which remained unstained by the Evans blue. The heart was excised, and the left ventricle (LV) was isolated, weighed, and sliced into 4 transverse pieces from base to apex, the first cutter blade being positioned at the site of the coronary occlusion. The slices were weighed, and color digital images of both sides of each slice were obtained with a Power Shot S50 zoom digital camera (Canon, Tokyo, Japan) connected to a microscope (Leica M Z 75; Leica Microsystems, Rueil-Malmaison, France), using the Adobe Photoshop software (Adobe Systems, San Jose, Calif., USA). The slices were then incubated at 37° C. with buffered 1% 2,3,5-triphenyltetrazolium chloride (TTC) solution for 20 min. Viable myocardium, which contained dehydrogenases, reacted with TTC and was stained brick red, whereas any necrotic tissue remained unstained due to the lack of active enzymes. The tissue sections were then fixed in a buffered 10% formalin solution for 24 h before being photographed again to delineate the IS. The cross-sectional area, the lumen area, the AR (unstained by Evans blue), and IS (unstained by TTC) of the LV were outlined on each color image and quantified by a masked observer using the Scion Image software (Scion Image for Windows; http://www.scioncorp.com). The absolute weights of AR and IS were then calculated for each slice. The sum of the absolute weight values of AR and IS of the 3 ischemic slices of each heart was calculated and expressed as a percentage of the total weight of the slice. The ratio of IS to AR was calculated from these absolute weight evaluations and expressed as a percentage of AR.

Results

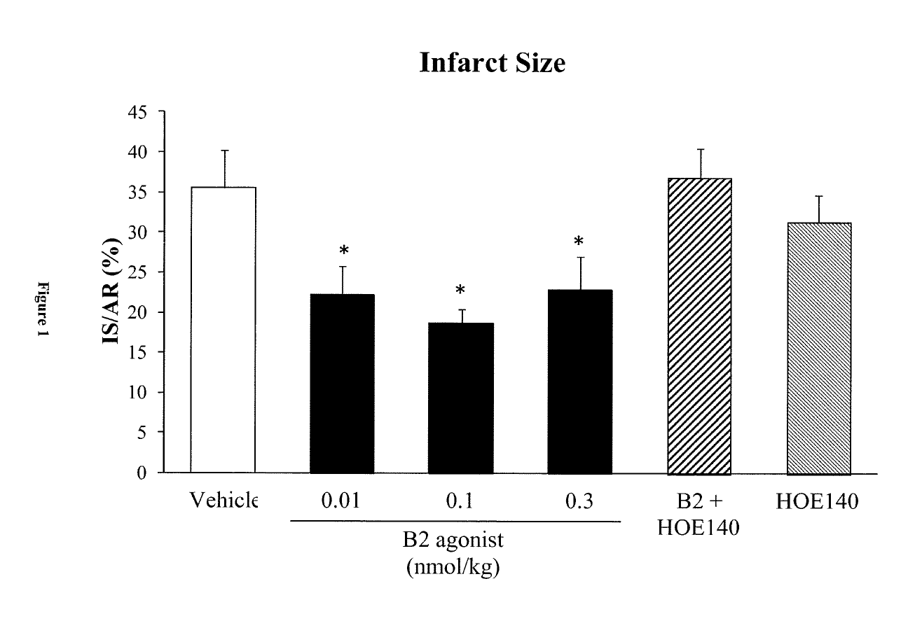

Heart rate remained unchanged throughout the IR experiments, and AR/LV ratios did not differ among the different experimental groups. B2 agonist injection just before the reperfusion, decreased the infarct size at each dose tested namely 0.01, 0.1 and 0.3 nmol/kg (−37.2%, −47.3%, −35.6% respectively, each p<0.05 vs saline) (FIG. 1). The B2 receptor antagonist HOE140 alone had no effect on infarct size and it suppressed the cardioprotective effect of B2 agonist.

### Example 2

### Effect of B2 Agonist in Peripheral Ischemia

Material and Methods

Ischemia Protocol:

Two-to three-month-old male C57BL/6J mice were used. Type 1 diabetes was induced by 5 daily ip injections of low-dose of streptozotocin (50 mg/kg dissolved in 0.05M sodium citrate buffer, pH 4.5). Ischemia of hinlimb was induced by permanent ligation of the right femoral artery once the diabetes is established (hyperglycemia >300 mg/dl, 4 weeks after streptozocin injections). Mice were anesthetized by isoflurane inhalation (0.8% in oxygen stream) and the proximal part of the femoral artery just below the origin of the circumflexa femoris lateralis was occluded with a silk suture. Concomitantly with the surgery, half the mice was treated with the B2 agonist (30 nmol/kg·h⁻¹), infused continuously through osmotic minipumps implanted subcutaneously. The other half of the mice was infused with the vehicle (isotonic saline). Healthy mice, non diabetic without ischemia were used as control.

Quantification of Neovascularization:

Two weeks after the onset of ischemia, neovascularization was evaluated by two methods. —Arteries were quantified by high definition microangiography using barium sulfate and digital X-ray transducer. Mice were anesthetized (Pentobarbital injection, 60 mg/kg, I.P.) and longitudinal laparatomy was performed to introduce a polyethylene catheter into the abdominal aorta to inject contrast medium (Barium sulfate, 1 mg/ml). Images (two per animal) were acquired using a high-definition digital X-ray transducer. Vessel density was expressed as a percentage of pixels per image occupied by vessel density in the quantification area. —Capillary density was analyzed by immunohistochemistry using fibronectin-FITC antibody. Frozen tissue sections (7 μm) from calf muscle were incubated with rabbit polyclonal antibody directed against total fibronectin (dilution 1:50) to identify capillaries. The number of capillary by field was determined in both ischemic and nonischemic legs. Results are expressed as ischemic to nonischemic ratio.

Results

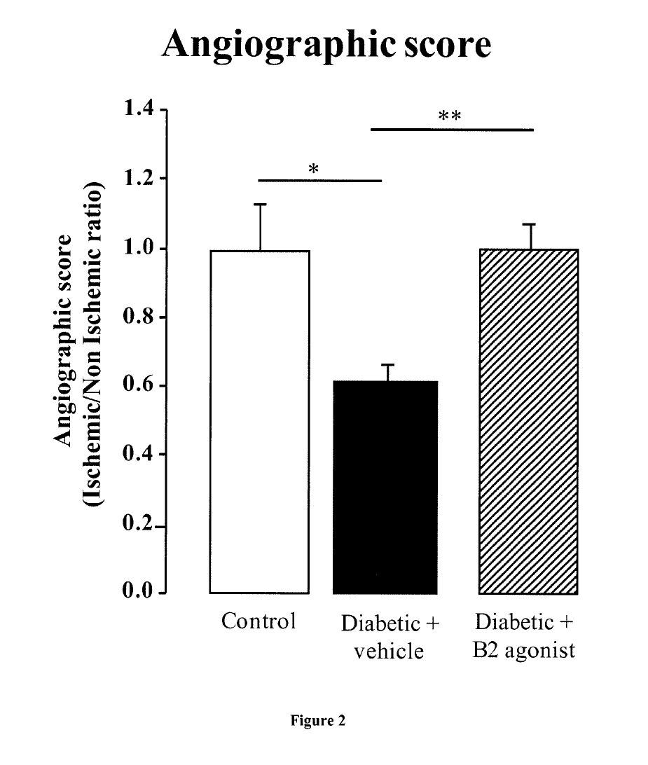

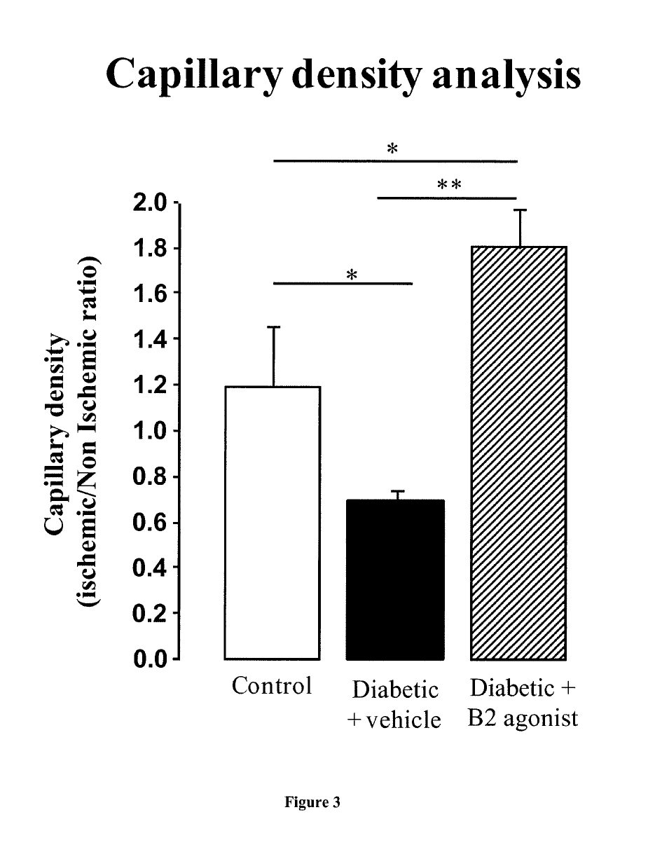

As expected, after 14 days of ischemia, diabetic mice present an important alteration of angiogenic process and a major decrease in limb blood supply, assessed by a significant decrease in angiographic score and capillary density when compared with non-diabetic mice. In contrast, diabetic mice treated with the B2 agonist showed an increase in angiographic score and capillary density, with values equivalent to those obtained in control, non-diabetic mice (FIGS. 2 and 3).

The results support the notion that synthetic chemically stable B2 agonists can be used therapeutically in the fields of cardiology and vascular diseases for treatment of a variety of acute and chronic cardiovascular diseases and syndromes, as well as in disturbances occuring in diabetes, in cerebrovascular accidents and in other diseases of arteries and peripheral vascular beds.

### REFERENCES

Throughout this application, various references describe the state of the art to which this invention pertains. The disclosures of these references are hereby incorporated by reference into the present disclosure.
Bélanger S, Bovenzi V, Côté J, Neugebauer W, Amblard M, Martinez J, Lammek B, Savard M, Gobeil F Jr. Peptides, 2009 April; 30(4):777-87.
Ebrahimian, T. G.; Tamarat, R.; Clergue, M.; Duriez, M.; Levy, B. I.; Silvestre, J. S. Arterioscler Thromb Vasc Biol, 2005, 25, 65-70.
Goto, M.; Liu, Y.; Yang, X. M.; Ardell, J. L.; Cohen, M. V.; Downey, J. M. Circ Res, 1995, 77, 611-21.
Griol-Charhbili V, Messadi-Laribi E, Bascands J L, Heudes D, Meneton P, Giudicelli J F, Alhenc-Gelas F, Richer C. FASEB J, 2005 July; 19(9):1172-4.
Hartman, J. C.; Wall, T. M.; Hullingcr, T. G.; Shebuski, R. J. J Cardiovasc Pharmacol, 1993, 21, 996-1003.
Leeb-Lundberg, L. M.; Marceau, F.; Muller-Esterl, W.; Pettibone, D. J.; Zuraw, B. L. Pharmacol Rev, 2005, 57, 27-77.
Regoli, D.; Rizzi, A.; Perron, S. I.; Gobeil, F., Jr. Biol Chem, 2001, 382, 31-5.
Silvestre, J. S.; Bergaya, S.; Tamarat, R.; Duriez, M.; Boulanger, C. M.; Levy, B. I. Circ Res, 2001, 89, 678-83.
Yang, X. P.; Liu, Y. H.; Scicli, G. M.; Webb, C. R.; Carretero, O. A. Hypertension, 1997, 30, 735-40.

1. A method of preventing or treating ischemia related organ damage in a subject in need thereof, comprising,
administering to said subject a therapeutically effective amount of a B2-receptor agonist.

2. The method according to claim 1 wherein said ischemia related organ damage is cardiac ischemia or ischemia-reperfusion organ damage.

3. The method according to claim 1 wherein said ischemia related organ damage is limb ischemia related organ damage.

4. The method according to claim 1 wherein said ischemia related organ damage is retinal ischemia related organ damage.

5. The method according to claim 1 wherein said ischemia related organ damage is kidney ischemia related organ damage.

6. The method according to claim 1 wherein said ischemia related organ damage is peripheral or diabetic ischemia related organ damage.

7. The method according to claim 1, wherein said B2-receptor agonist is selected from the group consisting of peptides, pseudopeptides, non-peptide compounds, peptide mimetics, bradykinin derivatives, and modified bradykinins.

8. The method according to claim 7 wherein said peptide is selected from the group consisting of kinin, lysyl-bradykinin, bradykinin analogue er and truncated bradykinin.

9. A pharmaceutical composition for use in the prevention and treatment of ischemia related organ damage in a subject in need thereof comprising a B2-receptor agonist and a pharmaceutical acceptable carrier.

10. A method of screening a candidate compound for use as a drug for the prevention or treatment of ischemia related organ damage in a subject in need thereof, wherein the method comprises the steps of:
providing a B2-receptor, providing a cell, tissue sample or organism expressing a B2-receptor,
providing a candidate compound selected from the group consisting of small organic molecules, peptides, polypeptides, peptide mimetics, metabolically and/or conformationally stabilized peptide analogs, derivatives or pseudo-peptides,
measuring the B2-receptor activity,
and selecting positively candidate compounds that induce B2-receptor activity.

11. The method according to claim 7 wherein said peptide is peptide 20 [Hyp(3), Thi(5), (N)Chg(7), Thi(8)]-BK.
